(19) United States
(12) Patent Application Publication
(10) Pub. No.: US 2017/0007660 A1
(43) Pub. Date: Jan. 12, 2017

(54) METHOD OF TREATING TINNITUS

(71) Applicant: Iraj E. Kiani, Newport Beach, CA (US)

(72) Inventor: Iraj E. Kiani, Newport Beach, CA (US)

(21) Appl. No.: 15/224,464

(22) Filed: Jul. 29, 2016

Related U.S. Application Data
(63) Continuation-in-part of application No. 14/740,189, filed on Jun. 15, 2015, now abandoned.

(51) Int. Cl. A61K 36/88; A61K 36/185

(52) U.S. Cl. CPC A61K 36/88; A61K 36/185

(57) ABSTRACT

A composition and a method of treating tinnitus comprising the composition and administration of onion extract, either by itself, in an effective delivery solution, or in combination with other active ingredients, such as sesame seed oil.

### CROSS-REFERENCES TO RELATED APPLICATIONS

This application is a continuation-in-part of U.S. patent application Ser. No. 14/740,189 filed Jun. 15, 2015, which is incorporated in its entirety hereby by this reference.

### TECHNICAL FIELD

This invention relates to compositions and methods for treating tinnitus through administration of bioactive agents.

### BACKGROUND

Tinnitus is a condition of perceiving sound when no external sound is present, the most common of which is a “ringing” noise. It may be a result of many different causes, including noise-induced hearing loss, ear infections, inflammation, foreign objects in the ear, nasal allergies, exposure to loud sounds, neurological damage, and quite possibly other presently unknown causes.

Tinnitus is a common disorder, with studies showing that between 25 and 50 million in the U.S. alone suffer from the disease. It is estimated to affect between 10% to 15% of the population, and there is no registered cure, to Applicant's knowledge. While many people eventually become used to living with this disorder, it can also severely impair the quality of life of a percentage of those afflicted.

Tinnitus is difficult to gauge objectively, as most of the symptoms are subjective. Identification and treatment of an underlying cause may eventually lead to improvements in tinnitus. As of 2014, however, it appears that no medication that has proven to be effective for tinnitus, and thus none are recommended.

For the foregoing reasons there is a need for a method of treating tinnitus.

### SUMMARY

The present invention is directed to a composition and method for treating tinnitus comprising the composition and administration of onion extract, either by itself, in an effective delivery solution, or in combination with other active ingredients, such as sesame seed oil.

### DETAILED DESCRIPTION OF THE INVENTION

The detailed description set forth below in connection is intended as a description of presently-preferred embodiments of the invention and is not intended to represent the only forms in which the present invention may be constructed or utilized. The description sets forth the functions and the sequence of steps for constructing and operating the invention. It is to be understood, however, that the same or equivalent functions and sequences may be accomplished by different embodiments that are also intended to be encompassed within the spirit and scope of the invention.

The present invention is directed towards the treatment of tinnitus with a composition comprising onion extract, either by itself, in an effective delivery solution, or in combination with other active ingredients. Such a treatment method would reduce or eliminate the perception of sounds when no external sound is present and/or other effects of such a condition.

One embodiment of the composition comprises a therapeutically effective amount of onion extract. The term “onion extract” as used herein shall mean extracts from, primarily, the bulb of plants in the onion family of plants that includes, but are not limited to, all species of onion, such as green onions, white onions, brown onions, yellow onions, red onions, sweet onions, etc., and specifically, the species Allium cepa (common onion), Allium fistulosum (Japanese bunching onion), Allium x proliferum (Egyptian onion), Allium canadense (Canada onion), and hybrids or combinations thereof, as well as other types of onion.

Onions are composed of various compounds that have medicinal value. These chemical compounds include, without limitation, allul propyle disulfide (allyl sulfide), Cis-3, trans-3, 5-diethyl, 1,2,4-trithiolene, anthocyanin, quercetin, and/or phytochemical compounds:

Generally Accepted Chemical Composition of Onion
Nutritive Value per 100 g of Onion

| Particulars | Big Onion (1) | Small Onion (1) | Onion Stalks (1) |
|---|---|---|---|
| Moisture (g) | 86.6 | 84.3 | 87.6 |
| Protein (g) | 1.2 | 1.8 | 0.9 |
| Fat (g) | 0.1 | 0.1 | 0.2 |
| Minerals (g) | 0.4 | 0.6 | 0.8 |
| Fibre (g) | 0.6 | 0.6 | 1.6 |
| Carbohydrate (g) | 11.1 | 12.6 | 8.9 |
| Energy (K cal) | 50.0 | 59.0 | 41.0 |
| Calcium (mg) | 46.9 | 40.0 | 50.0 |
| Phosphorus (mg) | 50.6 | 60.0 | 50.0 |
| Iron (mg) | 0.6 | 1.2 | 7.43 |
| Carotene (ug) | — | 15.0 | 595.0 |
| Thiamin (mg) | 0.08 | 0.08 | — |
| Riboflavin (mg) | 0.01 | 0.02 | 0.03 |
| Niacin (mg) | 0.4 | 0.5 | 0.3 |
| Folic acid (Total) | 6.0 | — | — |
| (mg) | | | |
| Vitamin C | 11.0 | 2.0 | 17.0 |
| Magnesium (mg) | 16.0 | — | 104.0 |
| Sodium (mg) | 4.0 | — | 2.2 |
| Potassium (mg) | 127.0 | — | 109.0 |
| Copper (mg) | 0.18 | — | 0.45 |
| Manganese (mg) | 0.18 | — | 0.74 |
| Molybdenum | 0.03 | — | 2.29 |
| (mg) | | | |
| Zinc (mg) | 0.41 | — | — |

While many health professionals recommend eating raw onions for maximum benefit, many people object to properties of raw onions, such as the taste, smell, or tear-inducing compounds. Thus, for maximum benefit and convenience, the beneficial components of an onion are extracted and, in some embodiments, treated to remove such objectionable properties. In some embodiments, the onion extract is cooled or combined with pleasant aromas to reduce or remove the smell of onions.

The onion extract may be present in the amount of anywhere between approximately 1% and approximately 99% by weight of the total composition. In another embodiment, the amount of onion extract may be between approximately 5% and approximately 99% by weight of the total composition. In another embodiment, the amount of onion extract may be between approximately 1% to approximately 95% by weight of the total composition. In an alternate embodiment, the amount of onion extract may be between approximately 5% and approximately 85% by weight of the total composition. In another embodiment, the onion extract the amount of onion extract may be between approximately 20% and approximately 80% by weight of the total composition. In another embodiment, the onion extract the amount of onion extract may be approximately 50% and approximately 75% by weight of the total composition. In some embodiments, the composition may be 100% onion extract.

In certain embodiments, this invention is directed towards a method of treating tinnitus comprising administering a therapeutically effective amount of a composition comprising onion extract, whereby the tinnitus is ameliorated.

The administration step may be any method of drug delivery, for example, ear drops, topical application, oral administration, patch application, rinse solution, parenterally, such as by subcutaneous injection, or the like. Most typically, the composition is administered via ear drops directly into the ear canal. Generally, one or two drops of the composition are sufficient per application. The composition preferably is applied for a period of time before flushing. In some embodiments, the composition may be administered one or more times a day. In some embodiments, the composition is administered daily. In some embodiments, the composition may be administered once a week, twice a week, or more.

Since administration as an ear drop is the preferred mode of administration, it is important to use onion extracts to be able to get the therapeutically effective amount of onion extract into the ear. For example, onion extract is at least two (2) times more concentrated than the onion juices occurring in nature. In some embodiments, onion extract can be at least five (5), ten (10), twenty (20), or fifty (50) times more concentrated than naturally occurring onion juice. Therefore, only a small volume of onion extract is required compared to onion juice. Due to the small volume of the ear canal, a therapeutically effective amount of the active ingredient may not be achieved using onion juice.

Additionally, as the concentration of the onion extract increases from 2 times that which is naturally occurring to five, ten, twenty, or fifty times the same, the viscosity likewise increases. As a result, the extract has the added benefit of being able to remain in the ear canal or at the eardrum longer than the comparably lower concentration, lower viscosity counterpart juices as they occur in nature. Therefore, in one embodiment, an effective treatment for tinnitus may comprise placing mere drops of a composition consisting essentially of onion extract concentrated as described above while allowing the patient to stand upright and move around without having a great fraction of the active ingredient falling away from the targeted region and inner surfaces of the ear canal.

In some embodiments, the composition for treating tinnitus may further comprise sesame seed oil. Sesame seed oil may provide at least two possible functions. First, sesame seed oil contains actives that may be effective in reducing tinnitus. For example, sesame seed oil contains vitamin K, oleic acid, linoleic acid, and palmitic acid. Any one or more of these compounds may contribute to ameliorating tinnitus or facilitate the onion extract in ameliorating tinnitus.

Second, as an oil, sesame seed oil can function as a carrier for the onion extract. Due to the narrow ear canal, and oil-based carrier will have a better chance of flowing through a narrow ear canal than a water-based carrier, which has a higher surface tension, especially given the fact that a small volume would have to be administered. Therefore, in one embodiment, an effective treatment for tinnitus may comprise placing drops of a composition consisting essentially of onion extract and sesame seed oil.

Therefore, sesame seed oil may make up approximately 1% to approximately 99% percent by weight of the total composition. In some embodiments, the sesame seed oil may make up approximately 1% to approximately 95% by weight of the total composition. In some embodiments, the sesame seed oil may make up approximately 5% to approximately 99% by weight of the total composition. In some embodiments, the sesame seed oil may make up approximately 15% to approximately 95% by weight of the total composition. In some embodiments, the sesame seed oil may make up approximately 20% to approximately 80% by weight of the total composition. In some embodiments, the sesame seed oil may make up approximately 25% to approximately 50% by weight of the total composition.

In use, a person suffering from tinnitus may be administered a composition comprising approximately 1% to approximately 99% by weight of onion extract and approximately 1% to approximately 99% by weight of sesame seed oil. In some embodiments, the composition may comprise approximately 5% to approximately 99% by weight of onion extract and approximately 1% to approximately 95% by weight of sesame seed oil. In another embodiment, the composition may comprise approximately 5% to approximately 85% by weight of onion extract and approximately 15% to approximately 95% by weight of sesame seed oil. In some embodiments, the composition may comprise approximately 20% to approximately 80% by weight of onion extract and approximately 20% to approximately 80% sesame seed oil. In some embodiments, the composition may comprise approximately 50% to approximately 75% by weight of onion extract and approximately 25% to approximately 50% by weight of sesame seed oil.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A method of treating tinnitus in a subject in need thereof, comprising: administering a therapeutically effective amount of a composition, comprising:
a. onion extract; and
b. sesame seed oil, wherein the tinnitus is ameliorated.

2. The method of claim 1, wherein the composition comprises, by weight of the composition, approximately 1% to approximately 99% of the onion extract; and approximately 1% to approximately 99% of the sesame seed oil.

3. The method of claim 1, wherein the composition comprises, by weight of the composition, approximately 5% to approximately 99% of the onion extract; and approximately 1% to approximately 95% of the sesame seed oil.

4. The method of claim 1, wherein the composition comprises, by weight of the composition, approximately 5% to approximately 85% of the onion extract; and approximately 15% to approximately 95% of the sesame seed oil.

5. The method of claim 1, wherein the composition comprises, by weight of the composition, approximately 20% to approximately 80% of the onion extract; and approximately 20% to approximately 80% of the sesame seed oil.

6. The method of claim 1, wherein the composition comprises, by weight of the composition, approximately 50% to approximately 75% of the onion extract; and approximately 25% to approximately 50% of the sesame seed oil.

7. The method of claim 1, wherein the composition is administered topically into an ear as an ear drop.

8. The method of claim 7, wherein the composition comprises, by weight of the composition, approximately 1% to approximately 99% of the onion extract; and approximately 1% to approximately 99% of the sesame seed oil.

9. The method of claim 7, wherein the composition comprises, by weight of the composition, approximately 5% to approximately 99% of the onion extract; and approximately 1% to approximately 95% of the sesame seed oil.

10. The method of claim 7, wherein the composition comprises, by weight of the composition, approximately 5% to approximately 85% of the onion extract; and approximately 15% to approximately 95% of the sesame seed oil.

11. The method of claim 7, wherein the composition comprises, by weight of the composition, approximately 20% to approximately 80% of the onion extract; and approximately 20% to approximately 80% of the sesame seed oil.

12. The method of claim 7, wherein the composition comprises, by weight of the composition, approximately 50% to approximately 75% of the onion extract; and approximately 25% to approximately 50% of the sesame seed oil.

13. The method of claim 1, wherein the composition is administered parenterally.

14. The method of claim 13, wherein the composition comprises, by weight of the composition, approximately 1% to approximately 99% of the onion extract; and approximately 1% to approximately 99% of the sesame seed oil.

15. The method of claim 13, wherein the composition comprises, by weight of the composition, approximately 5% to approximately 99% of the onion extract; and approximately 1% to approximately 95% of the sesame seed oil.

16. The method of claim 13, wherein the composition comprises, by weight of the composition, approximately 5% to approximately 85% of the onion extract; and approximately 15% to approximately 95% of the sesame seed oil.

17. The method of claim 13, wherein the composition comprises, by weight of the composition, approximately 20% to approximately 80% of the onion extract; and approximately 20% to approximately 80% of the sesame seed oil.

18. The method of claim 13, wherein the composition comprises, by weight of the composition, approximately 50% to approximately 75% of the onion extract; and approximately 25% to approximately 50% of the sesame seed oil.
